(19) United States
(12) Patent Application Publication
(10) Pub. No.: US 2006/0121387 A1
(43) Pub. Date: Jun. 8, 2006

(54) Toner processes

(75) Inventors: Edward Graham Zwartz, Mississauga (CA); T. Brian McAneney, Burlington (CA)

Correspondence Address: Marylou J. Lavoie, Esq. LLC, 1 Banks Road, Simsbury, CT, 06070, US

(73) Assignee: Xerox Corporation

(21) Appl. No.: 11/003,966

(22) Filed: Dec. 3, 2004

(51) Int. Cl. G03G 15/06; A61L 2/00

(52) U.S. Cl. 430/120; 422/22

(57) ABSTRACT

A process comprises developing an image on a document having a toner composition applied to the document, wherein the toner composition comprises a resin substantially free of cross linking, a cross linked resin, a wax, and a colorant; and wherein the developed document possesses the characteristic of resistance to adverse effects of electron beam irradiation. In embodiments, the process further includes, during fusing, migrating the wax and cross linked resin to the surface of the toner particles thereby imparting protection to the toner particles against exposure to elevated temperatures.

### RELATED APPLICATIONS

Commonly assigned, co-pending U.S. Patent Application of Raj D. Patel, Daryl Vanbesien, Enno E. Agur, Edward G. Zwartz, Maria N. V. McDougall, Emily L. Moore, Patricia A. Burns, Kimberly D. Nosella, Kelly Zhou, Vladislav Skorokhod, Wafa F. Bashir, and Shigang Steven Qui, Ser. No. ______, Attorney Docket Number A3307-US-NP, entitled “Toner Compositions,” filed of even date herewith, which is hereby incorporated by reference herein in its entirety, describes toner compositions and processes, such as, for example, emulsion aggregation toner processes for preparing a toner comprising a resin substantially free of cross linking, a cross linked resin, a wax, and a colorant.

Commonly assigned, co-pending U.S. Patent Application of Edward Graham Zwartz, T. Brian McAneney, Daryl Vanbesien, Patricia Burns, and Hwee Ng, Ser. No. ______, Attorney Docket Number A3310-US-NP, entitled “Toner Compositions,” filed of even date herewith, which is hereby incorporated by reference herein in its entirety, describes toner compositions comprising a non cross linked resin, a cross linked resin, a wax, a colorant, and a coagulant providing a toner having desirable characteristics such as excellent fusing characteristics.

Commonly assigned, co-pending U.S. Patent Application of Vladislav Skorokhod, Wafa Faisul Bashir, Maria N. V. McDougall, and Shigang Steven Qiu, Ser. No. ______, Attorney Docket Number 20031314-US-NP, entitled “Toner Compositions,” filed of even date herewith, which is hereby incorporated by reference herein in its entirety, describes toner compositions comprising a non cross linked resin; a cross linked resin; a wax; and a conductive colorant, wherein the compositions have optimized colorant loadings to provide excellent image quality in combination with alleviation or elimination of undesirable effects associated with inductive charging.

Commonly assigned, co-pending U.S. Patent Application of Maria N. V. McDougall and Richard P. N. Veregin, Ser. No. ______, Attorney Docket Number DA2370, entitled “Toner Compositions” filed of even date herewith, which is hereby incorporated by reference herein in its entirety, describes a toner composition comprising a binder, colorant, and a charge control surface additive mixture comprising a mixture of a first titanium dioxide possessing a first conductivity and a second titanium dioxide possessing a second conductivity and which second conductivity is dissimilar from the first conductivity; wherein the mixture of the first titanium dioxide and the second titanium dioxide is selected in a ratio sufficient to impart a selected triboelectric charging characteristic to the toner composition.

The appropriate components and process aspects of each of the foregoing may be selected for the present toner process in embodiments thereof

### TECHNICAL FIELD

The present disclosure relates to toner processes and more particularly relates to processes for developing a document with a toner composition which renders the image on the developed document resistant to adverse effects of electron beam irradiation.

### BACKGROUND

Beginning in the fall of 2001, it became standard practice to have mail (internal and external) destined for government offices in the Washington, D.C., area irradiated by electron beams at levels sufficient to destroy anthrax spores. General information on electron beam irradiation and its impact on materials can be found, for example, on the Smithsonian Center for Materials Research and Education website (http://www.si.edu/scmre/, general website address; http://www.si.edu/scmre/about/mail_irradiation.htm, article entitled “The effects on research specimens and museum collection items from electron beam irradiation of mail by the United States Postal Service”). The Smithsonian Center website provides information on the interaction of radiation and materials, radiation effects on materials, and the consequences of electron beam irradiation of United States Postal Service (USPS) mail for museum and archival collections items. The amount of irradiation applied to USPS mail for protection against anthrax spores is estimated to be about 25 to about 40 KiloGrays. Information on mail irradiation can also be found, for example, on the following websites: the Health Physics Society website (http://hps.org/publicinformation/ate/q1723.html); the United States Postal Service website (http://www.usps.com); the Organic Consumers Association website (http://www.organicconsumers.org), and IBA (Ion Beam Applications) website (http://www.iba.be/).

A by-product of this irradiation process is heat. Temperatures of the irradiated materials can reach about 130° C. Further, unique conditions are present during the process; that is, high temperatures and low pressures. The high temperature environment generated by the irradiation process is capable of melting compact disks, floppy disks, and other plastics. Irradiated documents become yellow and the paper becomes quite brittle. The high temperature environment further causes xerographic prints to stick to letters, other prints, and envelopes, etc. In some cases, the xerographic printed document is destroyed during the process of peeling apart the stuck documents. Image quality degradation of irradiated mail documents includes color changes such as yellowing. Mechanical damage to printed materials includes materials becoming stuck together, possibly due to the softening of the resins in the printing inks or photocopying toner. Moisture driven from the paper by high temperature can also condense and cause “blocking”. Government agencies and businesses that send large volumes of mail to the Washington, D.C. area have a need for equipment that can produce prints capable of surviving such electron beam irradiation while maintaining high image quality of the prints.

For both black and color prints, a small particle size toner is known to improve the image quality of the prints. High speed black and white printers require toner particles that can provide a matte finish in an oil-less fuser system with a low minimum fixing temperature (MFT) to enable high speed printing and at the same time achieve superior image quality in the resultant printed product.

### REFERENCES

U.S. Pat. No. 6,447,974 describes in the Abstract a process for the preparation of a latex polymer by (i) preparing or providing a water aqueous phase containing an anionic surfactant in an optional amount of less than or equal to about 20 percent by weight of the total amount of anionic surfactant used in forming the latex polymer; (ii) preparing or providing a monomer emulsion in water which emulsion contains an anionic surfactant; (iii) adding about 50 percent or less of said monomer emulsion to said aqueous phase to thereby initiate seed polymerization and to form a seed polymer, said aqueous phase containing a free radical initiator; and (iv) adding the remaining percent of said monomer emulsion to the composition of (iii) and heating to complete an emulsion polymerization thus forming a latex polymer.

U.S. Pat. No. 6,413,692 describes in the Abstract a process comprising coalescing a plurality of latex encapsulated colorants and wherein each of said encapsulated colorants are generated by miniemulsion polymerization.

U.S. Pat. No. 6,309,787 describes in the Abstract a process comprising aggregating a colorant encapsulated polymer particle containing a colorant with colorant particles and wherein said colorant encapsulated latex is generated by a miniemulsion polymerization.

U.S. Pat. No. 6,294,306 describes in the Abstract toners which include one or more copolymers combined with colorant particles or primary toner particles and a process for preparing a toner comprising (i) polymerizing an aqueous latex emulsion comprising one or more monomers, an optional nonionic surfactant, an optional anionic surfactant, an optional free radical initiator, an optional chain transfer agent, and one or more copolymers to form emulsion resin particles having the one or more copolymers dispersed therein; (ii) combining the emulsion resin particle with colorant to form statically bound aggregated composite particles; (iii) heating the statically bound aggregated composite particles to form toner; and (iv) optionally isolating the toner.

U.S. Pat. No. 6,130,021 describes in the Abstract a process involving the mixing of a latex emulsion containing resin and a surfactant with a colorant dispersion containing a nonionic surfactant, and a polymeric additive and adjusting the resulting mixture pH to less than about 4 by the addition of an acid and thereafter heating at a temperature below about, or equal to about, the glass transition temperature (Tg) of the latex resin, subsequently heating at a temperature above about, or about equal to, the Tg of the latex resin, cooling to about room temperature, and isolating the toner product.

U.S. Pat. No. 5,928,830 describes in the Abstract a process for the preparation of a latex comprising a core polymer and a shell thereover and wherein the core polymer is generated by (A) (i) emulsification and heating of the polymerization reagents of monomer, chain transfer agent, water, surfactant, and initiator; (ii) generating a seed latex by the aqueous emulsion polymerization of a mixture comprised of part of the (i) monomer emulsion, from about 0.5 to about 50 percent by weight, and a free radical initiator, and which polymerization is accomplished by heating, and, wherein the reaction of the free radical initiator and monomer produces a seed latex containing a polymer; (iii) heating and adding to the formed seed particles of (ii) the remaining monomer emulsion of (I), from about 50 to about 99.5 percent by weight of monomer emulsion of (i) and free radical initiator; (iv) whereby there is provided said core polymer; and (B) forming a shell thereover said core generated polymer and which shell is generated by emulsion polymerization of a second monomer in the presence of the core polymer, which emulsion polymerization is accomplished by (i) emulsification and heating of the polymerization reagents of monomer, chain transfer agent, surfactant, and an initiator; (ii) adding a free radical initiator and heating; (iii) whereby there is provided said shell polymer.

U.S. Pat. No. 5,869,558 describes in the Abstract dielectric black particles for use in electrophoretic image displays, electrostatic toner or the like, and the corresponding method of manufacturing the same. The black particles are latex particles formed by a polymerization technique, wherein the latex particles are stained to a high degree of blackness with a metal oxide.

U.S. Pat. No. 5,869,216 describes in the Abstract a process for the preparation of toner comprising blending an aqueous colorant dispersion and a latex emulsion containing resin; heating the resulting mixture at a temperature below about the glass transition temperature (Tg) of the latex resin to form toner sized aggregates; heating said resulting aggregates at a temperature above about the Tg of the latex resin to effect fusion or coalescence of the aggregates; redispersing said toner in water at a pH of above about 7; contacting the resulting mixture with a metal halide or salt, and then with a mixture of an alkaline base and a salicylic acid, a catechol, or mixtures thereof at a temperature of from about 25 degrees Celsius to about 80 degrees Celsius; and optionally isolating the toner product, washing, and drying.

U.S. Pat. No. 6,576,389 describes in the Abstract a process for the preparation of toner by, for example, mixing a colorant, a latex, a wax and a dual coagulant mixture comprising water solubilized silica with an alumina coating referred to as aluminized silica and a polyaluminum chloride to provide, for example, a toner composition of different gloss levels when fused. Additional patents of interest include U.S. Pat. No. 5,766,818; U.S. Pat. No. 5,344,738; and U.S. Pat. No. 4,291,111.

The disclosures of each of the United States Patents recited herein are totally incorporated herein by reference in their entireties. The appropriate components and process aspects of each of the patents recited herein may be selected for the present process in embodiments thereof.

There remains a need for an improved toner composition and process which further overcomes or alleviates the above-described and other problems experienced in the art. There is specifically a need for a toner and toner process suitable for use in xerographic engines that can produce quality images which images can survive irradiation processes without adverse effect on the printed document.

### SUMMARY

Illustrated herein is a process comprising developing an image on a document with a toner composition comprising a resin substantially free of cross linking, a cross linked resin, a wax, and a colorant; and wherein the developed document possesses the characteristic of being protected from or resistant to the adverse effects of electron beam irradiation. A unique combination of toner materials allows the developed toner to survive electron beam (e-beam) irradiation at irradiation levels sufficient to destroy anthrax spores. In embodiments, the process comprises treating the developed document with electron beam irradiation; and wherein the developed document possesses the characteristic of being substantially free of offset damage after the treating with electron beam irradiation.

In embodiments, the process comprises treating the developed document with electron beam irradiation at an irradiation level sufficient to destroy anthrax spores. In further embodiments, the process provides treating the developed document with electron beam irradiation at an irradiation level of about 25 Kilo Grays to about 40 Kilo Grays.

In embodiments, the toner composition comprises a high glass transition temperature (Tg) resin substantially free of cross linking (also referred to as a non cross linked resin) wherein high glass transition temperature comprises a Tg in the range of about 46° C. to about 62° C., or about 58° C., although not limited and wherein substantially free of cross linking means a resin having substantially zero percent cross linking to less than about 0.1 percent cross linking. In embodiments, a cross linked resin or gel comprises a cross linked resin or gel having for example about 0.3 percent to about 20 percent cross linking. In embodiments, the toner composition comprises a high Tg resin substantially free of cross linking, a cross linked resin, a wax, and a colorant, wherein the composition and quantity of the high Tg resin substantially free of cross linking, the cross linked resin, and the wax in combination provide toner particles that are resistant to adverse effects of electron beam irradiation.

Aspects further include preparing a toner by an emulsion aggregation process comprising mixing the resin substantially free of cross linking and the cross linked resin in the presence of the wax, the colorant, and coagulant and heating to a temperature below the resin Tg of the resin substantially free of cross linking to provide toner size aggregates; adding additional resin substantially free of cross linking to the formed aggregates thereby providing a shell over the formed aggregates; heating the shell covered aggregates to a temperature above the resin Tg of the resin substantially free of cross linking to form toner; and optionally, isolating the toner. The composition and quantity of the high Tg non cross linked resin, the cross linked resin or gel and the wax in combination provide toner particles that are resistant to adverse effects of electron beam irradiation.

The toner composition alleviates or eliminates altogether problems associated with electron beam irradiation of toner printed documents, such as xerographic toner sticking to paper and to itself

In aspects, the present toner processes are suitable for use in xerographic engines and produce quality images which survive irradiation processes without adverse effect on the printed document. The toner processes provide in embodiments a toner composition and process having low melt fusing properties in combination with excellent image robustness. The toner compositions and processes include small particle size toner with high pigment loading allowing less toner to be placed onto a document while still maintaining image quality. The significantly lower amounts of toner allow large cost savings to be achieved. While not being bound by theory, it is believed that the combination of high Tg resin for document offset in combination with cross linked resin or gel for reducing gloss and wax for releasing and preventing stripper finger mark damage in toner particles enables xerograph prints produced with the toner composition to survive high temperatures, for example, temperatures of about 90° C. to about 130° C. at very low loads for short periods of time. Prints thus produced do not stick together after mail irradiation treatment or, if stuck, are easily separated without apparent damage to the document.

These and other features and advantages will be more fully understood from the following description of certain specific embodiments taken together with the accompanying claims.

### DESCRIPTION

The present toner process comprises in embodiments a process comprising developing an image on a document having a toner composition applied to the document, wherein the toner composition comprises a resin substantially free of cross linking, a cross linked resin or gel, a wax, and a colorant; and wherein the developed document possesses the characteristic of resistance to adverse effects of electron beam irradiation. The resin substantially free of cross linking is in embodiments a high glass transition temperature (Tg) resin and in combination with the cross linked resin or gel; the wax, and colorant provide a toner suitable for use in xerographic engines that can produce quality images and survive irradiation processes without adverse effect on the printed document. In embodiments, further provided is a process for preparing a toner comprising mixing a high Tg resin substantially free of cross linking and a cross-linked resin or gel in the presence of a wax, colorant, and coagulant to provide toner size aggregates; adding additional high Tg resin substantially free of cross linking to the formed aggregates thereby providing a shell over the formed aggregates; heating the shell covered aggregates to form toner; and, optionally, isolating the toner. Further provided, in embodiments, are irradiation resistant documents produced with the present process which documents are substantially free of offset damage after exposure to electron beam irradiation.

### Latex Resins or Polymers

Illustrative examples of latex resins or polymers selected for the non cross linked resin and cross linked resin or gel include for example known polymers such as poly(styrene-butadiene), poly(methyl styrene-butadiene), poly(methyl methacrylate-butadiene), poly(ethyl methacrylate-butadiene), poly(propyl methacrylate-butadiene), poly(butyl methacrylate-butadiene), poly(methyl acrylate-butadiene), poly(ethyl acrylate-butadiene), poly(propyl acrylate-butadiene), poly(butyl acrylate-butadiene), poly(styrene-isoprene), poly(methyl styrene-isoprene), poly(methyl methacrylate-isoprene), poly(ethyl methacrylate-isoprene), poly(propyl methacrylate-isoprene), poly(butyl methacrylate-isoprene), poly(methyl acrylate-isoprene), poly(ethyl acrylate-isoprene), poly(propyl acrylate-isoprene), poly(butyl acrylate-isoprene); poly(styrene-propyl acrylate), poly(styrene-butyl acrylate), poly(styrene-butadiene-acrylic acid), poly(styrene-butadiene-methacrylic acid), poly(styrene-butyl acrylate-acrylic acid), poly(styrene-butyl acrylate-methacrylic acid), poly(styrene-butyl acrylate-acrylonitrile), poly(styrene-butyl acrylate-acrylonitrile-acrylic acid), and the like or mixtures thereof. In embodiments, the resin or polymer is a styrene/butyl acrylate/carboxylic acid terpolymer.

### High Tg Resin

For example, high Tg resin substantially free of cross linking comprises a high Tg resin having an onset glass transition temperature (Tg) in the range of about 46° C. to about 62° C. or about 58° C., although not limited.

For example, the high Tg resin substantially free of cross linking comprises in embodiments a styrene/butyl acrylate/carboxylic acid terpolymer. In embodiments, the high Tg resin substantially free of cross linking comprises styrene, butylacrylate, and beta-carboxy ethyl acrylate (beta-CEA) monomers, although not limited to these monomers, termed herein as monomers A, B, and C, preferably prepared by emulsion polymerization in the presence of an initiator, a chain transfer agent (CTA), and surfactant.

Further examples of resin substantially free of cross linking are selected from the group consisting of, but not limited to, methyl acrylate, methyl methacrylate, ethyl acrylate, ethyl methacrylate, propyl acrylate, propyl methacrylate, butyl acrylate, butyl methacrylate, pentyl acrylate, pentyl methacrylate, hexyl acrylate, hexyl methacrylate, heptyl acrylate, heptyl methacrylate, octyl acrylate, octyl methacrylate, cyclohexyl acrylate, cyclohexyl methacrylate, lauryl acrylate, lauryl methacrylate, stearyl acrylate, stearyl methacrylate, benzyl acrylate, benzyl methacrylate, ethoxypropyl acrylate, ethoxypropyl methacrylate, methylbutyl acrylate, methylbutyl methacrylate, ethylhexyl acrylate, ethylhexyl methacrylate, methoxybutyl acrylate, methoxybutyl methacrylate, cyanobutyl acrylate, cyanobutyl methacrylate, tolyl acrylate, tolyl methacrylate, styrene, substituted styrenes, or mixtures thereof. In embodiments, the resin substantially free of cross linking is a styrene/butyl acrylate/carboxylic acid terpolymer.

In embodiments, the non cross linked resin monomers are present in an amount of about 70% to about 90% styrene, about 10% to about 30% butylacrylate, and about 0.05 parts per hundred to about 10 parts per hundred beta-CEA, or about 3 parts per hundred beta-CEA, by weight based upon the total weight of the monomers and wherein the total of said components is about 100%, although not limited. For example, the carboxylic acid is not limited and can be selected from the group comprising of acrylic acid, methacrylic acid, itaconic acid, beta carboxy ethyl acrylate (beta CEA), fumaric acid, maleic acid, and cinnamic acid, and wherein a carboxylic acid is selected in an amount of from about 0.05 to about 10 weight percent based upon the weight of the resin substantially free of cross linking.

In embodiments, the high Tg non cross linked resin comprises for example styrene:butylacrylate:beta-CEA. For example, the high Tg non cross linked resin comprises about 73% to about 85% styrene, about 27% to about 15% butylacrylate, and about 1.0 part per hundred to about 5 parts per hundred beta-CEA, by weight based upon the total weight of the monomers although the compositions and processes are not limited to these particular types of monomers or ranges. In another feature, the high Tg non cross linked resin comprises about 81.7% styrene, about 18.3% butylacrylate and about 3.0 parts per hundred beta-CEA by weight based upon the total weight of the monomers.

The initiator may be, for example, but is not limited to, sodium or ammonium persulfate and is present in embodiments in the range of about 0.5 to about 3.0 percent by weight based upon the weight of the monomers. For example, the CTA is present in an amount of from about 1.5 to about 3.0 percent by weight based upon the combined weight of the monomers A and B. The surfactant is, for example, an anionic surfactant present in the range of about 0.7 to about 5.0 percent by weight based upon the weight of the aqueous phase, although not limited to this type or range.

In aspects, the monomers are polymerized under starve fed conditions as referred to in Xerox patents such as U.S. Pat. No. 6,447,974, U.S. Pat. No. 6,576,389, U.S. Pat. No. 6,617,092, and U.S. Pat. No. 6,664,017, each of which are hereby incorporated by reference herein in their entireties, to provide latex resin particles having a size in the range of about 100 to about 300 nanometers in size.

The molecular weight of the high Tg resin is in embodiments from about 30,000 to about 37,000, or about 34,000, although not limited to these ranges.

In embodiments, the amount of carboxylic acid groups is selected in the range of about 0.04 to about 4.0 parts per hundred of the resin monomers A and B.

The Mn of the high Tg resin is in embodiments from about 5,000 to about 20,000, or about 11,000, although not limited.

In embodiments, the prepared high Tg resin has a pH of about 1.0 to about 4.0, or about 2.0.

### Cross Linked Resin or Gel

An exemplary cross linked latex is prepared from a non cross linked latex comprising styrene, butylacrylate, beta-CEA, and divinyl benzene, termed herein as monomers A, B, C, and D, by emulsion polymerization, in the presence of an initiator such as a persulfate, a CTA, and surfactant. In embodiments, the cross linked resin monomers are present in a ratio of about 60% to about 75% styrene, about 40% to about 25% butylacrylate, about 3 parts per hundred to about 5 parts per hundred beta-CEA, and about 3 parts per hundred to about 5 parts per hundred divinyl benzene, although not limited to these particular types of monomers or ranges. For example, the monomer composition may comprise, for example, about 65% styrene, 35% butylacrylate, 3 parts per hundred beta-CEA, and about 1 parts per hundred divinyl benzene, although the composition is not limited to these amounts.

The Tg (onset) of the cross linked latex is, in embodiments about 40° C. to about 55° C. or about 42° C. and the degree of cross linking is, in embodiments, in the range of about 2 to about 20 percent, although not limited to these parameters, since, for example, an increase in the divinyl benzene concentration will increase the cross linking.

The soluble portion of the cross linked latex has in embodiments a Mw of about 135,000 and a Mn of about 27,000 but is not limited thereto.

In embodiments, the particle size of the cross linked latex is in the range of about 50 to about 150 nanometers or about 50 nanometers, although not limited.

The surfactant may be any surfactant, such as, but not limited to, an anionic surfactant such as Neogen RK. In embodiments, the pH of the latex is about 1.8.

The latex particle size can be, for example, from about 0.05 micron to about 1 micron in average volume diameter as measured by the Brookhaven nanosize particle analyzer. Other sizes and effective amounts of latex particles may be selected in embodiments. In embodiments, the latex resins selected are prepared by emulsion polymerization methods, and the monomers utilized in such processes include in embodiments the monomers listed above, such as, styrene, acrylates, methacrylates, butadiene, isoprene, acrylonitrile, acrylic acid, and methacrylic acid, and beta CEA. Known chain transfer agents, for example dodecanethiol in effective amounts of, for example, from about 0.1 to about 10 percent, and/or carbon tetrabromide in effective amounts of from about 0.1 to about 10 percent, can also be employed to control the resin molecular weight during the polymerization.

Other processes of obtaining resin particles of from, for example, about 0.05 micron to about 1 micron can be selected from polymer microsuspension process, such as the processes disclosed in U.S. Pat. No. 3,674,736, the disclosure of which is totally incorporated herein by reference, polymer solution microsuspension processes, such as disclosed in U.S. Pat. No. 5,290,654, the disclosure of which is totally incorporated herein by reference, mechanical grinding processes, or other known processes.

### Surfactants

Surfactants in amounts of, for example, from about 0.01 to about 20, or more specifically from about 0.1 to about 15 weight percent of the reaction mixture in embodiments include, for example, nonionic surfactants such as dialkylphenoxypoly(ethyleneoxy) ethanol, available from Rhone-Poulenc as IGEPAL CA-210.™., IGEPAL CA-520.™., IGEPAL CA-720.™., IGEPAL CO-890.™., IGEPAL CO-720.™., IGEPAL CO-290.™., IGEPAL CA-210.™., ANTAROX 890.™. and ANTAROX 897.™. An effective concentration of the nonionic surfactant is in embodiments, for example, from about 0.01 to about 10 percent by weight, or from about 0.1 to about 5 percent by weight of the reaction mixture.

Examples of anionic surfactants being, for example, sodium dodecylsulfate (SDS), sodium dodecylbenzene sulfonate, sodium dodecylnaphthalene sulfate, dialkyl benzenealkyl, sulfates and sulfonates, adipic acid, available from Aldrich, NEOGEN R.™., NEOGEN SC.™. available from Kao, and the like. An effective concentration of the anionic surfactant generally employed is, for example, from about 0.01 to about 10 percent by weight, or from about 0.1 to about 5 percent by weight of the reaction mixture

Examples of bases used to increase the pH and hence ionize the aggregate particles thereby providing stability and preventing the aggregates from growing in size can be selected from sodium hydroxide, potassium hydroxide, ammonium hydroxide, cesium hydroxide and the like.

Examples of the additional surfactants, which may be added optionally to the aggregate suspension prior to, or during the coalescence to, for example, prevent the aggregates from growing in size, or for stabilizing the aggregate size, with increasing temperature can be selected from anionic surfactants such as sodium dodecylbenzene sulfonate, sodium dodecylnaphthalene sulfate, dialkyl benzenealkyl, sulfates and sulfonates, adipic acid, available from Aldrich, NEOGEN R.™., NEOGEN SC.™. available from Kao, and the like. These surfactants can also be selected from nonionic surfactants such as polyvinyl alcohol, polyacrylic acid, methalose, methyl cellulose, ethyl cellulose, propyl cellulose, hydroxy ethyl cellulose, carboxy methyl cellulose, polyoxyethylene cetyl ether, polyoxyethylene lauryl ether, polyoxyethylene octyl ether, polyoxyethylene octylphenyl ether, polyoxyethylene oleyl ether, polyoxyethylene sorbitan monolaurate, polyoxyethylene stearyl ether, polyoxyethylene nonylphenyl ether, dialkylphenoxypoly(ethyleneoxy) ethanol, available from Rhone-Poulenac as IGEPAL CA-210.™., IGEPAL CA-520.™., IGEPAL CA-72.™., IGEPAL CO-890.™., IGEPAL CO-720.™., IGEPAL CO-290.™., IGEPAL CA-210.™., ANTAROX 890.™. and ANTAROX 897.™. An effective amount of the anionic or nonionic surfactant generally employed as an aggregate size stabilization agent is, for example, from about 0.01 to about 10 percent by weight, or from about 0.1 to about 5 percent by weight of the reaction mixture.

Examples of the acids that can be utilized include, for example, nitric acid, sulfuric acid, hydrochloric acid, acetic acid, citric acid, trifluro acetic acid, succinic acid, salicylic acid and the like, and which acids are utilized in a diluted form in the range of about 0.5 to about 10 weight percent by weight of water or in the range of about 0.7 to about 5 weight percent of water.

### Wax

For example, wax suitable for the present toner compositions include, but are not limited to, alkylene waxes having about 1 to about 25 carbon atoms including, for example, polyethylene, polypropylene or mixtures thereof. Examples of waxes include those as illustrated herein, such as those of the aforementioned co-pending applications, polypropylenes and polyethylenes commercially available from Allied Chemical and Petrolite Corporation, wax emulsions available from Michaelman Inc. and the Daniels Products Company, Epolene N-15™ commercially available from Eastman Chemical Products, Inc., Viscol 550-P™, a low weight average molecular weight polypropylene available from Sanyo Kasei K.K., and similar materials. The commercially available polyethylenes possess, it is believed, a molecular weight Mw of from about 1,000 to about 5,000, and the commercially available polypropylenes are believed to possess a molecular weight of from about 4,000 to about 10,000. Examples of functionalized waxes include amines, amides, for example Aqua Superslip 6550™, Superslip 6530™ available from Micro Powder Inc., fluorinated waxes, for example Polyfluo 190™, Polyfluo 200™, Polyfluo 523XF™, Aqua Polyfluo 411™, Aqua Polysilk 19™, Polysilk 14™ available from Micro Powder Inc., mixed fluorinated, amide waxes, for example Microspersion 19™ also available from Micro powder Inc., imides, esters, quaternary amines, carboxylic acids or acrylic polymer emulsion, for example Joncryl 74™, 89™, 130™, 537™, and 538™, all available from SC Johnson Wax, chlorinated polypropylenes and polyethylenes available from Allied Chemical and Petrolite Corporation and SC Johnson Wax.

In embodiments, the wax comprises a wax dispersion comprising a wax having a particle diameter of about 100 to about 500 nanometers, water, and an anionic surfactant. The wax is included in amounts such as from about 3% to about 30% or about 5% to about 15% by weight percent based upon the total weight of the composition. The wax comprises in embodiments polyethylene wax particles, such as Polywax 850™, commercially available from Baker Petrolite, although not limited thereto, having a particle diameter in the range of about 100 to about 500 nanometers. The surfactant used to disperse the wax is in embodiments an anionic surfactant, although not limited thereto, such as, for example, Neogen RK™ commercially available from Kao Corporation.

In embodiments, the process contemplates selectively locating the wax at different locations in the toner particle and providing different wax loadings to achieve optimum toner performance. In embodiments, the process comprises employing a toner particle having a surface, the toner particle comprising the compositions described herein; and wherein the wax is disposed on or near the surface of the toner particle or no greater than about 1 micron below the surface of the toner particle. In alternate embodiments, the process comprises employing a toner particle comprising the compositions described herein; and wherein the wax is distributed throughout the toner particle.

Bulk addition and delayed wax processes such as have been described in U.S. Pat. Nos. 5,910,389; 6,333,131, 6,340,549, 6,416,920, 6,576,389, incorporated by reference in their entireties hereinabove, may be employed in the present process.

### Bulk Addition Process

In embodiments, the toner is prepared having the wax distributed throughout the toner particles. In embodiments, the toner composition and process include a wax content of about 3% to about 15%, or about 5% to about 11% or about 9% by weight based on the total weight of the toner composition using a bulk wax addition process (Toner sample 9, below).

### Delayed Wax Process

In embodiments, the wax and gel are disposed close to the surface of the toner particles using a delayed wax process. For example, in embodiments, the wax is disposed on or near the surface of the toner particle or no more than about 1 micron below the surface of the toner particle. This allows less wax to be used thereby lowering costs while maintaining performance. For example, in embodiments, the toner composition and process include a wax content of 5% by weight based on the total weight of the toner composition using a delayed wax addition process. See toner sample 8, below, for example.

In embodiments, the process further includes, during fusing, migrating the wax and cross linked resin or gel to the surface of the toner particles thereby imparting protection to the toner particles against exposure to elevated temperatures.

### Pigment/Colorant

Examples of colorants or pigments suitable for the present toner process include, but are not limited to, pigment, dye, mixtures of pigment and dye, mixtures of pigments, mixtures of dyes, and the like. For simplicity, the term “colorant” as used herein is meant to encompass-such colorants, dyes, pigments, and mixtures, unless specified as a particular pigment. For example, the colorant comprises in embodiments a pigment, a dye, carbon black, magnetite, black, cyan, magenta, yellow, red, green, blue, brown, or mixtures thereof, in an amount of about 1% to about 25% by weight based upon the total weight of the composition. It is to be understood that other useful colorants will become readily apparent to one of skill in the art based on the present disclosures.

In embodiments, the colorant comprises a black pigment. In further embodiments the pigment is carbon black. In still further embodiments, the pigment comprises black toner particles having a shape factor of about 120 to about 140 where a shape factor of 100 is considered to be spherical and a circularity of about 0.900 to about 0.980 as measured on an analyzer such as a Sysmex FPIA 2100 analyzer, where a circularity of 1.00 is considered to be spherical in shape.

In further embodiments, the colorant comprises a carbon black pigment dispersion, such as Regal 300™ commercially available, prepared in an anionic surfactant and optionally a non-ionic dispersion to provide pigment particles having a size of from about 50 nanometers to about 300 nanometers. The surfactant used to disperse the carbon black is in embodiments an anionic surfactant such as Neogen RK, although not limited thereto. An ultimizer type equipment is used to provide the pigment dispersion, although media mill or other means can also be used.

Optionally, other various known colorants such as dyes or pigments may be present in the toner in an effective amount of, for example, about 1 percent to about 65 percent, or in an amount of about 1 percent to about 25 percent, or about 1 percent to about 15 percent, or about 3 to about 10 percent, by weight based upon the weight of the toner composition. In general, useful colorants or pigments in addition to carbon black include magnetite, or mixtures thereof; cyan, yellow magenta, or mixtures thereof; or red, green, blue, brown, or mixtures thereof. Colorants that may be used include, but are not limited to, Paliogen Violet 5100 and 5890 (BASF), Normandy Magenta RD-2400 (Paul Uhlrich), Permanent Violet VT2645 (Paul Uhlrich), Heliogen Green L8730 (BASF), Argyle Green XP-111-S (Paul Uhlrich), Brilliant Green Toner GR 0991 (Paul Uhirich), Lithol Scarlet D3700 (BASF), Toluidine Red (Aldrich), Scarlet for Thermoplast NSD Red (Aldrich), Lithol Rubine Toner (Paul Uhlrich), Lithol Scarlet 4440, NBD 3700 (BASF), Bon Red C (Dominion Color), Royal Brilliant Red RD-8192 (Paul Uhlrich), Oracet Pink RF (Ciba Geigy), Paliogen Red 3340 and 3871K (BASF), Lithol Fast Scarlet L4300 (BASF), Heliogen Blue D6840, D7080, K7090, K6910 and L7020 (BASF), Sudan Blue OS (BASF), Neopen Blue FF4012 (BASF), PV Fast Blue B2G01 (American Hoechst), Irgalite Blue BCA (Ciba Geigy), Paliogen Blue 6470 (BASF), Sudan II, III and IV (Matheson, Coleman, Bell), Sudan Orange (Aldrich), Sudan Orange 220 (BASF), Paliogen Orange 3040 (BASF), Ortho Orange OR 2673 (Paul Uhlrich), Paliogen Yellow 152 and 1560 (BASF), Lithol Fast Yellow 0991K (BASF), Paliotol Yellow 1840 (BASF), Novaperm Yellow FGL (Hoechst), Permanerit Yellow YE 0305 (Paul Uhlrich), Lumogen Yellow D0790 (BASF), Suco-Gelb 1250 (BASF), Suco-Yellow D1355 (BASF), Sico Fast Yellow D 165, D1355 and D1351 (BASF), Hostaperm Pink E (Hoechst), Fanal Pink D4830 (BASF), Cinquasia Magenta (DuPont), Paliogen Black L9984 9BASF), Pigment Black K801 (BASF) and particularly carbon blacks such as REGAL 330™, Carbon Black 5250™ and 5750™ (Columbian Chemicals), and the like or mixtures thereof.

Additional useful colorants include pigments in water based dispersions such as those commercially available from Sun Chemical, for example SUNSPERSE BHD 6011X (Blue 15 Type), SUNSPERSE BHD 9312X (Pigment Blue 15 74160), SUNSPERSE BHD 6000X (Pigment Blue 15:3 74160), SUNSPERSE GHD 9600× and GHD 6004X (Pigment Green 7 74260), SUNSPERSE QHD 6040X (Pigment Red 122 73915), SUNSPERSE RHD 9668X (Pigment Red 185 12516), SUNSPERSE RHD 9365X and 9504X (Pigment Red 57 15850:1, SUNSPERSE YHD 6005X (Pigment Yellow 83 21108), FLEXIVERSE YFD 4249 (Pigment Yellow 17 21105), SUNSPERSE YHD 6020X and 6045X (Pigment Yellow 74 11741), SUNSPERSE YHD 600X and 9604X (Pigment Yellow 14 21095), FLEXIVERSE LFD 4343 and LFD 9736 (Pigment Black 7 77226) and the like or mixtures thereof. Other useful water based colorant dispersions include those commercially available from Clariant, for example, HOSTAFINE Yellow GR, HOSTAFINE Black T and Black TS, HOSTAFINE Blue B2G, HOSTAFINE Rubine F6B and magenta dry pigment such as Toner Magenta 6BVP2213 and Toner Magenta EO2 which can be dispersed in water and/or surfactant prior to use.

Other useful colorants include, for example, magnetites, such as Mobay magnetites MO8029, MO8960; Columbian magnetites, MAPICO BLACKS and surface treated magnetites; Pfizer magnetites CB4799, CB5300, CB5600, MCX6369; Bayer magnetites, BAYFERROX 8600, 8610; Northern Pigments magnetites, NP-604, NP-608; Magnox magnetites TMB-100 or TMB-104; and the like or mixtures thereof. Specific additional examples of pigments include phthalocyanine HELIOGEN BLUE L6900, D6840, D7080, D7020, PYLAM OIL BLUE, PYLAM OIL YELLOW, PIGMENT BLUE 1 available from Paul Uhlrich & Company, Inc., PIGMENT VIOLET 1, PIGMENT RED 48, LEMON CHROME YELLOW DCC 1026, E.D. TOLUIDINE RED and BON RED C available from Dominion Color Corporation, Ltd., Toronto, Ontario, NOVAPERM YELLOW FGL, HOSTAPERM PINK E from Hoechst, and CINQUASIA MAGENTA available from E.I. DuPont de Nemours & Company, and the like. Examples of magentas include, for example, 2,9-dimethyl substituted quinacridone and anthraquinone dye identified in the Color Index as CI 60710, CI Dispersed Red 15, diazo dye identified in the Color Index as CI 26050, CI Solvent Red 19, and the like or mixtures thereof. Illustrative examples of cyans include copper tetra(octadecyl sulfonamide) phthalocyanine, x-copper phthalocyanine pigment listed in the Color Index as CI74160, CI Pigment Blue, and Anthrathrene Blue identified in the Color Index as DI 69810, Special Blue X-2137, and the like or mixtures thereof. Illustrative examples of yellows that may be selected include diarylide yellow 3,3-dichlorobenzidene acetoacetanilides, a monoazo pigment identified in the Color Index as CI 12700, CI Solvent Yellow 16, a nitrophenyl amine sulfonamide identified in the Color Index as Foron Yellow SE/GLN, CI Dispersed Yellow 33 2,5-dimethoxy-4-sulfonanilide phenylazo-4′-chloro-2,4-dimethoxy acetoacetanilide, and Permanent Yellow FGL. Colored magnetites, such as mixtures of MAPICOBLACK and cyan components may also be selected as pigments.

### Coagulant

In embodiments, the coagulants used in the present process comprise poly metal halides, such as polyaluminum chloride (PAC) or polyaluminum sulfo silicate (PASS). In embodiments, the coagulants provide a final toner having a metal content of about 400 to about 10,000 parts per million. In embodiments, the coagulant comprises a poly aluminum chloride providing a final toner having an aluminum content of about 400 to about 10,000 parts per million.

### Preparation of High Tg Non-Cross Linked Resin

An exemplary high Tg non-cross linked latex comprising styrene, butylacrylate, and beta-carboxy ethyl acrylate (beta-CEA) monomers, termed herein as monomers A, B, and C, is prepared by emulsion polymerization in the presence of an initiator, a chain transfer agent (CTA), and surfactant. In aspects, the composition of the monomers is in embodiments 76.5% styrene, 23.5% butyl acrylate, and 3.0 parts per hundred beta-CEA, although not limited to these particular types of monomers or ranges. The initiator may be, for example, but is not limited to, sodium or ammonium persulfate, and is present in embodiments in the range of about 0.5 to about 3.0 percent based upon the weight of the monomers. The CTA is present in embodiments in an amount of from about 1.5 to about 3.0 percent by weight based upon the combined weight of the monomers A and B. The surfactant is in embodiments an anionic surfactant present in the range of about 0.7 to about 5.0 percent by weight based upon the weight of the aqueous phase, although not limited to this type or range. In embodiments, the monomers are polymerized under starve fed conditions as referred to in Xerox patents such as U.S. Pat. No. 5,444,140 and U.S. Pat. No. 5,455,315, which are hereby incorporated by reference herein in their entireties, to provide latex resin particles having a size in the range of about 100 to about 300 nanometers in size. The latex resin particles comprise about 76.5% styrene (A), about 23.5% butylacrylate (B), by weight and about 3 parts per hundred beta-CEA (C). The molecular weight of the latex resin about 30,000 to about 37,000 although not limited to this range. The onset glass transition temperature (TG) is not limited, however, but may be in the range, for example, of from about 46° C. to about 62° C. or about 58° C. The amount of carboxylic acid groups is in the range of about 0.05 to about 4.0 parts per hundred of the resin monomers A and B. In embodiments, the onset Tg of the polymer resin obtained is about 58° C., although not limited thereto, the Mw is about 34,000, and the Mn is about 11,000, although not limited thereto, to provide a non-cross linked latex having a pH of about 2.0.

### Preparation of Cross Linked Latex Gel

In aspects, an exemplary cross linked latex is prepared from a non-cross linked latex comprising styrene, butylacrylate, beta-CEA, and divinyl benzene, termed herein as monomers A, B, C, and D, by emulsion polymerization, in the presence of an initiator such as a persulfate, a CTA, and surfactant. The monomer composition comprises in embodiments 65% styrene, 35% butylacrylate, 3 parts per hundred beta-CEA, and 1 part per hundred divinyl benzene, although the composition is not limited to these amounts. The Tg (onset) of the cross linked latex is about 42° C. and the degree of cross linking is in the range of about 2 to about 20 percent, although not limited thereto, since an increase in the divinyl benzene concentration will increase the cross linking. The soluble portion of the cross linked latex has in embodiments a Mw of about 135,000 and a Mn of about 27,000, but is not limited thereto. The particle size of the cross linked latex is about 50 nanometers, although not limited, and can be in the range of about 50 to about 150 nanometers. The surfactant may be any surfactant, such as, but not limited to, an anionic surfactant such as Neogen RK. The pH of the latex is about 1.8.

### Preparation of Carbon Black Dispersion

A carbon black dispersion is prepared such as with Regal 300™, commercially available, in an anionic surfactant and optionally a non-ionic dispersion to provide pigment particles having a size of from about 50 nanometers to about 300 nanometers. The surfactant used to disperse the carbon black is in embodiments an anionic surfactant such as Neogen RK, although not limited thereto. Preferably, an ultimizer type equipment is used to provide the pigment dispersion, although media mill or other means can also be used.

### Emulsion Aggregation Toner Particle Preparation

Emulsion/aggregation/coalescing processes for the preparation of toners are illustrated in a number of Xerox patents, the disclosures of each of which are totally incorporated herein by reference, such as U.S. Pat. No. 5,290,654, U.S. Pat. No. 5,278,020, U.S. Pat. No. 5,308,734, U.S. Pat. No. 5,370,963, U.S. Pat. No. 5,344,738, U.S. Pat. No. 5,403,693, U.S. Pat. No. 5,418,108, U.S. Pat. No. 5,364,729, and U.S. Pat. No. 5,346,797. Also of interest are U.S. Pat. Nos. 5,348,832; 5,405,728; 5,366,841; 5,496,676; 5,527,658; 5,585,215; 5,650,255; 5,650,256; 5,501,935; 5,723,253; 5,744,520; 5,763,133; 5,766,818; 5,747,215; 5,827,633; 5,853,944; 5,804,349; 5,840,462; 5,869,215; 5,863,698; 5,902,710; 5,910,387; 5,916,725; 5,919,595; 5,925,488; and 5,977,210, the disclosures of each of which are totally incorporated herein by reference. In addition, Xerox U.S. Pat. Nos. 6,627,373; 6,656,657; 6,617,092; 6,638,677; 6,576,389; 6,664,017; 6,656,658; and 6,673,505 are each totally incorporated herein by reference. The appropriate components and process aspects of each of the foregoing may be selected for the present process in embodiments thereof.

In embodiments, the toner process comprises forming an emulsion aggregation toner particle by mixing the high Tg non cross linked latex with a quantity of the cross linked latex in the presence of a wax and pigment dispersion to which is added a coagulant of a poly metal halide such as polyaluminum chloride while blending at high speeds such as with a polytron. The resulting mixture having a pH of about 2.0 to about 3.0 is aggregated by heating to a temperature below the resin Tg to provide toner size aggregates. Additional non cross linked latex is added to the formed aggregates providing a shell over the formed aggregates. The pH of the mixture is then changed by the addition of a sodium hydroxide solution until a pH of about 7.0 is achieved. When the mixture reaches a pH of about 7.0, the carboxylic acid becomes ionized to provide additional negative charge on the aggregates thereby providing stability and preventing the particles from further growth or an increase in the grain size distribution when heated above the Tg of the latex resin. The temperature of the mixture is then raised to about 95° C. After about 30 minutes, the pH of the mixture is reduced to a value sufficient to coalesce or fuse the aggregates to provide a composite particle upon further heating such as about 4.5. The fused particles are measured for shape factor or circularity, such as with a Sysmex FPIA 2100 analyzer, until the desired shape is achieved.

The mixture is allowed to cool to room temperature and is washed. A first wash is conducted such as at a pH of about 10 and a temperature of about 63° C. followed by a deionized water (DIW) wash at room temperature. This is followed by a wash at a pH of about 4.0 at a temperature of about 40° C. followed by a final DIW water wash. The toner is then dried.

In aspects of the present process, the toner composition comprising a high Tg non cross linked latex, a cross linked latex, a wax, and a colorant, the cross linked latex is primarily used to increase the hot offset, while the wax is used to provide release characteristics. The ratio of the high Tg non cross linked latex to the cross linked latex, the wax content and the colorant content are selected to control the rheology of the toner.

The toner comprises in embodiments about 68% to about 75% high Tg non cross linked resin (resin substantially free of cross linking), about 6% to about 13% cross linked resin, about 6% to about 12% wax, and about 7% to about 13% colorant, by weight, based upon the total weight of the composition wherein the total of said components is about 100%, although not limited thereto.

In embodiments, the toner composition comprises 71 percent non cross linked resin, 10 weight percent cross linked resin, 9 weight percent wax, and 10 weight percent colorant, a shape factor of about 120 to about 140, and a particle circularity of about 0.900 to about 0.980.

In embodiments, the toner process comprises, during fusing, migrating the wax and cross linked resin or gel to the surface of the toner particles thereby imparting protection to the toner particles against exposure to elevated temperatures.

The following examples are set forth as. These examples are not to be construed as limiting in scope as these and other equivalent embodiments will be apparent in view of the present disclosure and accompanying claims.

### EXAMPLES

### Toner

The toners 1-9 of Table 1 were prepared according to the following procedures and having the following compositions.

Toner 1: Commercially available conventional toner for Xerox iGen3 Production Printer.

Toner 2: Commercially available conventional toner for NexPress Production Printer.

Toner 3: Conventional toner for Xerox Highlight Color Printer.

Toner 4: Commercially available conventional toner for Xerox DocuTech Printers.

Toner 5: Commercially available conventional toner for Xerox DC555 Printer.

Toner 6: Experimental chemical toner prepared by the Emulsion Aggregation Toner Particle Preparation procedures described hereinabove.

Toner 7: Commercially available conventional toner for Xerox Nuvera 100 Printer.

Toner 8: Experimental chemical toner prepared by the Emulsion Aggregation Toner Particle Preparation procedures described hereinabove for Xerox Monochrome Printers.

Toner 9: Experimental chemical toner prepared by the Emulsion Aggregation Toner Particle Preparation procedures described hereinabove for Xerox Monochrome Printers.

### Examples 1-9

For each toner 1-9, a sample document 1-9 was printed on an uncoated and coated paper substrate using the printer indicated in Table 1. The printed samples were placed in standard letter size, greeting card size and magazine size envelopes to determine which xerographic prints would survive electron beam irradiation with no damage or minimal damage.

The envelopes containing samples 1-9 were placed in cardboard boxes and sent through an irradiation facility in aluminum trays. The boxes were irradiated twice, once at the top and once at the bottom at a dose of approximately 40 kilo Gray each time in accordance with USPS standard irradiation procedure. The process comprises passing the boxes containing the samples through the irradiation chamber, with each pass taking approximately 20 minutes. The boxes are then turned and ran through the irradiation chamber a second time. The actual irradiation time for each pass is estimated about approximately 20 seconds. The temperature during irradiation was measured using temperature indicating tape included in several of the sample envelopes. The temperature was determined to be between about 93° C. and 110° C.

TABLE 1

| Example/Toner | Printer | Tg | Gel | Wax | DO | Comment |
|---|---|---|---|---|---|---|
| 1 | Xerox iGen3 Production | Low | No | No | Red | Severe blocking. Sheets of paper |
| | Printer | | | | | tear apart when being separated. |
| 2 | NexPress Production | Low | No | No | Red | Severe blocking. Sheets of paper |
| | Priner | | | | | tear apart when being separated. |
| 3 | Xerox experimental | High | Yes | No | Yellow | Hilight colors sticking, some damage. |
| | High light Color Printer | | | | | |
| 4 | Xerox Docutech Printer | High | No | No | Red | Sticking. Significant damage. |
| 5 | Xerox DC555 Printer | High | No | Yes | Yellow | Sticking. Some damage. |
| 6 | Xerox experimental | Low | Yes | Yes | Yellow | Sticking. Some damage. |
| | Monochrome Printer | | | | | |
| 7 | Xerox Nuvera 100 Printer | High | Yes | Yes | Green | Little or no sticking. No damage. |
| 8 | Xerox experimental | High | Yes | Yes | Green | Little or no sticking. No damage. |
| | Monochrome Printer | | | | | |
| 9 | Xerox experimental | High | Yes | Yes | Green | Little or no sticking. No damage. |
| | Monochrome Printer | | | | | |

In the Table 1, a low Tg means a toner having an overall glass transition temperature in the range of about 48° C. to about 54° C.; DO indicates document offset, with the color designation indicating a DO as follows:

Red=Severe toner blocking and major damage to the print;

Yellow=Some toner blocking, visible damage to the print; and

Green=No toner blocking, very little or no visible damage to the print.

The printed samples of Examples 1-9 were visually evaluated. As shown in Table 1, the printed samples prepared with the present toner having in combination high Tg resin, gel and wax exhibit little or no sticking and no visible damage.

Examples 8 and 9 illustrate that a range in the amount of wax content of the xerographic toner is possible while still providing damage free or minimally damaged irradiated print. Example 7 having 9% bulk wax addition and Example 8 providing 5% delayed wax addition each produce documents exhibiting little or no sticking and no visible damage to the separated documents after irradiation.

The claims, as originally presented and as they may be amended, encompass variations, alternatives, modifications, improvements, equivalents, and substantial equivalents of the embodiments and teachings disclosed herein, including those that are presently unforeseen or unappreciated, and that, for example, may arise from applicants/patentees and others.

What is claimed is:

1. A process comprising:
developing an image on a document with a toner composition comprising a resin substantially free of cross linking, a cross linked resin, a wax, and a colorant; and
wherein the developed document possesses the characteristic of being protected from or resistant to the adverse effects of electron beam irradiation.

2. The process of claim 1, further comprising:
treating the developed document with electron beam irradiation; and
wherein the developed document possesses the characteristic of being substantially free of offset damage after the treating with electron beam irradiation.

3. The process of claim 1, wherein the electron beam irradiation comprises treating the developed document with electron beam irradiation at a level sufficient to destroy anthrax spores.

4. The process of claim 1, wherein the electron beam irradiation comprises treating the developed document with electron beam irradiation at an irradiation level of about 25 Kilo Grays to about 40 Kilo Grays.

5. The process of claim 1, including a toner particle comprising the toner composition of claim 1, the toner particle having a surface; and
wherein the wax is disposed on or near the surface of the toner particle.

6. The process of claim 1, including a toner particle comprising the toner composition of claim 1, the toner particle having a surface, and;
wherein the wax is disposed on the surface of the toner particle or no greater than about 1 micron below the surface of the toner particle.

7. The process of claim 1, including a toner particle comprising the toner composition of claim 1; and
wherein the wax is distributed throughout the toner particle.

8. The process of claim 1, further comprising:
during fusing, migrating the wax and cross linked resin or gel to the surface of the toner thereby imparting protection to the toner against exposure to elevated temperatures.

9. The process of claim 8, wherein elevated temperatures means a temperature of about 90° C. to about 130° C.

10. The process of claim 1, further comprising preparing a toner by an emulsion aggregation process comprising:
mixing the resin substantially free of cross linking and the cross linked resin in the presence of the wax, the colorant, and a coagulant and heating to a temperature below the resin Tg of the resin substantially free of cross linking to provide toner size aggregates;
adding additional resin substantially free of cross linking to the formed aggregates thereby providing a shell over the formed aggregates;
heating the shell covered aggregates to a temperature above the resin Tg of the resin substantially free of cross linking to form toner; and
optionally, isolating the toner.

11. The process of claim 1, wherein the toner composition comprises about 68% to about 75% resin substantially free of cross linking, about 6% to about 13% cross linked resin, about 6% to about 12% wax, and about 7% to about 13% colorant, by weight based upon the total weight of the composition and wherein the total of said components is about 100%.

12. The process of claim 1, wherein the toner composition possesses a particle circularity of about 0.900 to about 0.980.

13. The process of claim 1, wherein the toner composition possesses a shape factor of about 120 to about 140.

14. The process of claim 1, wherein at least one of the resin substantially free of cross linking and the cross linked resin are selected from the group consisting of poly(styrene-butadiene), poly(methyl styrene-butadiene), poly(methyl methacrylate-butadiene), poly(ethyl methacrylate-butadiene), poly(propyl methacrylate-butadiene), poly(butyl methacrylate-butadiene), poly(methyl acrylate-butadiene), poly(ethyl acrylate-butadiene), poly(propyl acrylate-butadiene), poly(butyl acrylate-butadiene), poly(styrene-isoprene), poly(methyl styrene-isoprene), poly(methyl methacrylate-isoprene), poly(ethyl methacrylate-isoprene), poly(propyl methacrylate-isoprene), poly(butyl methacrylate-isoprene), poly(methyl acrylate-isoprene), poly(ethyl acrylate-isoprene), poly(propyl acrylate-isoprene), and poly(butyl acrylate-isoprene); poly(styrene-propyl acrylate), poly(styrene-butyl acrylate), poly(styrene-butadiene-acrylic acid), poly(styrene-butadiene-methacrylic acid), poly(styrene-butyl acrylate-acrylic acid), poly(styrene-butyl acrylate-methacrylic acid), poly(styrene-butyl acrylate-acrylonitrile), poly(styrene-butyl acrylate-acrylonitrile-acrylic acid), styrene/butyl acrylate/carboxylic acid, or mixtures thereof.

15. The process of claim 1, wherein the resin substantially free of cross linking has an onset glass transition temperature in the range of about 46° C. to about 62° C.

16. The process of claim 1, wherein the resin substantially free of cross linking is selected from the group consisting of methyl acrylate, methyl methacrylate, ethyl acrylate, ethyl methacrylate, propyl acrylate, propyl methacrylate, butyl acrylate, butyl methacrylate, pentyl acrylate, pentyl methacrylate, hexyl acrylate, hexyl methacrylate, heptyl acrylate, heptyl methacrylate, octyl acrylate, octyl methacrylate, cyclohexyl acrylate, cyclohexyl methacrylate, lauryl acrylate, lauryl methacrylate, stearyl acrylate, stearyl methacrylate, benzyl acrylate, benzyl methacrylate, ethoxypropyl acrylate, ethoxypropyl methacrylate, methylbutyl acrylate, methylbutyl methacrylate, ethylhexyl acrylate, ethylhexyl methacrylate, methoxybutyl acrylate, methoxybutyl methacrylate, cyanobutyl acrylate, cyanobutyl methacrylate, tolyl acrylate, tolyl methacrylate, styrene, substituted styrenes, or mixtures thereof.

17. The process of claim 1, wherein the resin substantially free of cross linking is a styrene/butyl acrylate/carboxylic acid terpolymer.

18. The process of claim 1, wherein the resin substantially free of cross linking comprises about 70% to about 90% styrene, about 10% to about 30% butylacrylate, and about 0.5 parts per hundred to about 10 parts per hundred beta-carboxy ethyl acrylate, by weight based upon the total weight of the resin substantially free of cross linking and wherein the total of said components is about 100%.

19. The process of claim 1, wherein the resin substantially free of cross linking comprises carboxylic acid in an amount of about 0.05% to about 10% by weight based upon the weight of the resin substantially free of cross linking and wherein the total of said components is about 100%.

20. The process of claim 1, wherein the resin substantially free of cross linking comprises about 73% to about 85% styrene, about 15% to about 27% butylacrylate, and about 1.0 part per hundred to about 5 part per hundred beta-carboxy ethyl acrylate, by weight based upon the total weight of the resin substantially free of cross linking and wherein the total of said components is about 100%.

21. The process of claim 1, wherein the cross linked resin comprises styrene:butylacrylate:beta-carboxy ethyl acrylate:divinyl benzene.

22. The process of claim 1, wherein the cross linked resin comprises about 60% to about 75% styrene, about 40% to about 25% butylacrylate, about 3 parts per hundred to about 5 parts per hundred beta-carboxy ethyl acrylate, and about 3 parts per hundred to about 5 parts per hundred divinyl benzene, by weight based upon the total weight of the cross linked resin and wherein the total of said components is about 100%.

23. The process of claim 1, wherein the wax is an alkylene having about 1 to about 25 carbon atoms, polyethylene, polypropylene, or mixtures thereof.

24. The process of claim 1, wherein the wax is present in an amount of about 3% to about 30% by weight based upon the total weight of the composition and wherein the total of said components is about 100%.

25. The process of claim 1, wherein the wax is present in an amount of about 5% to about 15% by weight based upon the total weight of the composition and wherein the total of said components is about 100%.

26. The process of claim 1, wherein the wax comprises a wax dispersion comprising a wax having a particle diameter of about 100 to about 500 nanometers, water, and an anionic surfactant.

27. The process of claim 1, wherein the colorant comprises a pigment, a dye, carbon black, magnetite, black, cyan, magenta, yellow, red, green, blue, brown, or mixtures thereof, in an amount of about 1% to about 25% by weight based upon the total weight of the composition.

28. The process of claim 1, wherein the colorant is a black pigment.
